(19) United States
(12) Patent Application Publication
(10) Pub. No.: US 2009/0196928 A1
(43) Pub. Date: Aug. 6, 2009

(54) Biocompatible hydrogel compositions

(76) Inventor: Olexander Hnojewyi, Redwood City, CA (US)

Correspondence Address: Deniel D. Ryan;RYAN KROMHOLZ & MANION, S.C., Post Office Box 26618, Milwaukee, WI, 53226-0618, US

(21) Appl. No.: 12/384,699

(22) Filed: Apr. 8, 2009

Related U.S. Application Data
(63) Continuation of application No. 11/233,737, filed on Sep. 23, 2005, now abandoned.
Application No. 11/233,737 is a continuation-in-part of application No. 10/948,765, filed on Sep. 23, 2004, now abandoned.

(51) Int. Cl. A61K 9/14; A61K 31/727; A61K 39/395; A61P 7/02; A61P 17/02

(52) U.S. Cl. 424/486; 514/56; 424/130.1

(57) ABSTRACT

Compositions, instruments, systems, and methods are providing for creating families of materials having diverse therapeutic indications and possessing enhanced biocompatibility. One genus platform for the families includes a biocompatible synthetic electrophilic component mixed with a nucleophilic component. The electrophilic component can include a functionalized electrophilic poly (anhydride ester) material. The nucleophilic material can include a natural, autologous protein. The components, when mixed in a liquid state, react by cross-linking, forming a solid matrix composition, or hydrogel.

### RELATED APPLICATIONS

This application is a continuation of co-pending application Ser. No. 11/233,737 filed 23 Sep. 2005, which is a continuation-in-part of U.S. patent application Ser. No. 10/948,765, filed Sep. 23, 2004.

### FIELD THE INVENTION

The invention relates to biocompatible materials and additives that are formulated for biomedical applications.

### BACKGROUND OF THE INVENTION

Hydrogel compounds, e.g., those based upon poly(ethylene glycol) (PEG)—have been utilized in several biomedical fields, including dermatology, drug delivery systems, stem cell delivery systems, and bonding and coating systems. Generally, many current fields of study that are concerned with tissue and tissue manipulation have produced research and compounds directed towards compositions and methods incorporating PEG compounds.

Many hydrogel PEG compounds are made from purely synthetic components or from mixtures of synthetic components combined with human or animal proteins that are derived from pooled blood sources drawn from random donors. When these PEG compounds are used, biocompatible issues may arise, particular with respect to those patients that suffer from AIDS or whose immune systems are otherwise challenged when exposed to blood products other than their own. Accordingly, improvements in the biocompatibility of PEG compounds or in hydrogel compounds in general are still desired, to minimize problems associated with the use of purely synthetic compositions or compositions relying upon pooled blood products.

There is a continuing need to develop new compositions capable of forming in situ biocompatible hydrogel structures that offer improved therapeutic outcomes.

### SUMMARY OF THE INVENTION

A. Autologous Hydrogel Compositions

One aspect of the invention provides compositions, instruments, systems, and methods for creating families of materials having diverse therapeutic indications and possessing enhanced biocompatibility. The genus platform for the families includes a biocompatible synthetic electrophilic (i.e., electron withdrawing) component mixed with a nucleophilic (i.e., electron donating) component that includes a natural, autologous protein. By “autologous,” it is meant that the human or animal protein is derived from the same individual human or animal to which the solid matrix composition is to be applied.

The components, when mixed in a liquid state, react by cross-linking, forming a solid matrix composition, or hydrogel. By “cross-linking,” it is meant that the hydrogel composition contains intermolecular crosslinks and optionally intramolecular crosslinks as well, arising from the formation of covalent bonds. The term “hydrogel” or “hydrogel composition” refers to a state of matter comprising a cross-linked polymer network swollen in a liquid medium.

According to this aspect of the invention, the hydrogel transforms over time by physiologic mechanisms from a solid state back to a biocompatible liquid state, which can be cleared by the body. Depending upon the selection of polymer for the backbone material, the transformation can occur by hydrolysis of the polymer backbone, or by surface erosion of the polymer backbone, or by a combination of the two.

The electrophilic component and/or the nucleophilic component can include additive components, e.g., buffered solutions and/or nucleophilic materials. The additive components can affect the reactivity of the components, when mixed, in terms of reaction time and the resulting physical and mechanical characteristics of the composition.

The electrophilic component and/or the nucleophilic component can, alone or in combination with the additive components, include auxiliary components, e.g., fillers, plasticizers, and/or therapeutic agents. The auxiliary components affect the resulting physical and mechanical characteristics of the composition, and/or make possible the use of the composition for a desired therapeutic indication, e.g., void filling or drug delivery. The compositions, instruments, systems, and methods make possible the mixing of the compositions directly at or on the delivery site.

Because the nucleophilic component includes autologous blood or a component derived from autologous blood, contamination that may have previously occurred from a pooled blood source drawn from random donors is minimized. The compositions, instruments, systems, and methods make possible the treatment of patients with AIDS or with otherwise compromised immune systems. Likewise, the use of the patient's own blood or blood compound provides a more biocompatible system than systems that use a purely artificial medium. Also, since the nucleophilic part of the mixture is provided directly from the patient, raw material supplies and costs will be reduced. It will not be necessary to supply an outside source, such as from a pooled blood source, an animal blood source, or artificial developed albumin source, allowing for a more cost efficient system.

B. Poly(Anhydride) Compositions

Another aspect of the invention provides bio-erodable compositions, instruments, systems, and methods for creating families of materials having diverse therapeutic indications and possessing enhanced biocompatibility. The genus platform for the families includes a biocompatible synthetic component comprising a poly (anhydride ester) (PAE). The PAE component can be placed into solution for use by mixing with a non-aqueous solvent, for application as a coating to metal, plastic, or ceramic materials intended for implantation in human or animal tissue. Alternatively, the PAE component can be made to be water soluable and placed into solution for use by mixing with an aqueous solvent, for application as a cream or dressing on animal tissue.

Another aspect of the invention provides a biocompatible functionalized electrophilic component comprising PAE that is, in use, mixed with a nucleophilic component. By “functionalized electrophilic component comprising PAE” it is meant that the basic molecular segment or backbone of PAE is modified to generate or introduce a new reactive electrophilic functional group (e.g., a succinimidyl group) that is capable of undergoing reaction with another functional nucleophilic group (e.g., an amine group) to form a covalent bond. The functionalized electrophilic component comprising PAE can either be not water soluable or modified to be water soluable.

The nucleophilic component can include a synthetic component (e.g., chemically synthesized in the laboratory or industrially or produced using recombinant DNA technology) or a natural (i.e., naturally occurring) component, such as a protein. If desired, when the functionalized electrophilic PAE component is made water soluable, the nucleophilic component can include a natural, autologous protein, providing the features and benefits attributed to the first aspect of the invention, just described. The components, when mixed in a liquid state, react by cross-linking, forming a solid matrix composition, or hydrogel, as previously defined.

PAE materials are disclosed in International Publication Number 2004/045549, entitled “Medical Devices Employing Novel Polymers,” and U.S. patent application Ser. No. 10/861,881, filed Jun. 4, 2004 (Publication No. US 2005/0048121), which are incorporated herein by reference. It has been discovered that such materials can be functionalized to form one or more electrophilic groups that react with nucleophilic components and form hydrogel structures. Hydrogels based upon functionalized poly(anhydride esters) can exhibit greater mechanical strength and stability than PEG-based hydrogels. The surface of PAE hydrogels can remain stable within the body for longer periods of time, because they undergo degradation more by erosion at the surface than liquification of the entire backbone. This phenomenon will sometimes be called “bio-erosion.” Compounds or agents that are incorporated into the PAE backbone structure can be released by bio-erosion in a more controlled fashion to any site of the host body.

C. Biocompatible Hydrogel Compositions With Retardant Additive

According to another aspect of the invention, a hydrogel composition, instrument, system, and method can include an N-hydroxy-succinimide (NHS) compound as an additive component. It has been discovered that the presence of NHS retards the initial reaction of the electrophilic component with a given nucleophilic material, affecting the gelation time independent of buffering to affect the reaction pH.

D. Therapeutic Indications

The therapeutic indications for compositions that incorporate one or more aspects of the invention include: (i) collagen restoration/replacement (e.g., topical application or void filling by injection to fill wrinkles, or for biopsy sealing); (ii) drug delivery (e.g., the delivery of glucosamine and chondroitin sulfate into the spine area or other body regions); (iii) stem cell or growth factor delivery (e.g., the delivery of stem cells and/or growth factors into the spine area or other body regions); (iv) tissue sealants/adhesives; (v) the control of bleeding or fluid leakage in body tissue (e.g., lung sealing or hemostasis); (vi) tissue, muscle, and bone growth and regeneration; (vii) dermatology (e.g., topical cosmetic and therapeutic creams, shampoos, soaps, and oils); (vii) internal and external bonding and coating of tissue and instruments, e.g., coatings for burn victims, artificial skin, adhesion prevention, coatings on polymers, or coatings for implant devices such as, e.g., stents.

Other features and advantages of the various aspects of the inventions are set forth in the following specification and drawings, as well as being defined in the appended claims.

### BRIEF DESCRIPTION OF THE DRAWINGS

FIG. 1 is a diagrammatic view of a system for creating families of biocompatible materials having diverse therapeutic indications based upon a biomaterial platform that includes a biocompatible synthetic electrophilic component mixed with a nucleophilic component that includes a natural, autologous protein.

FIG. 2 is a view of a kit that can be used to deliver the system shown in FIG. 1.

FIG. 3A is a diagrammatic view of a system for creating families of biocompatible materials having diverse therapeutic indications based upon a biomaterial platform that includes a biocompatible poly(anhydride ester) material, which can either be placed into solution for use with a non-water-based solvent or be modified, if desired, so that it can be placed into solution for use with a water-based solvent.

FIG. 3B is a diagrammatic view of a system for creating families of biocompatible materials having diverse therapeutic indications based upon a biomaterial platform that includes a biocompatible and functionalized electrophilic poly(anhydride ester) material, which can be either water soluable or not, mixed with a nucleophilic component to form a hydrogel.

FIG. 4 is a microphotograph of dried human blood, which possesses brittle mechanical characteristics.

FIG. 5 is a microphotograph of a hydrogel structure comprising an electrophilic poly(ethylene glycol) (PEG) material mixed with autologous blood, demonstrating that the presence of PEG has transformed the brittle nature of dry blood into a robust physical structure that can adhere and conform to tissue with beneficial therapeutic results.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

### I. Autologous Hydrogel Compositions

A. System Overview

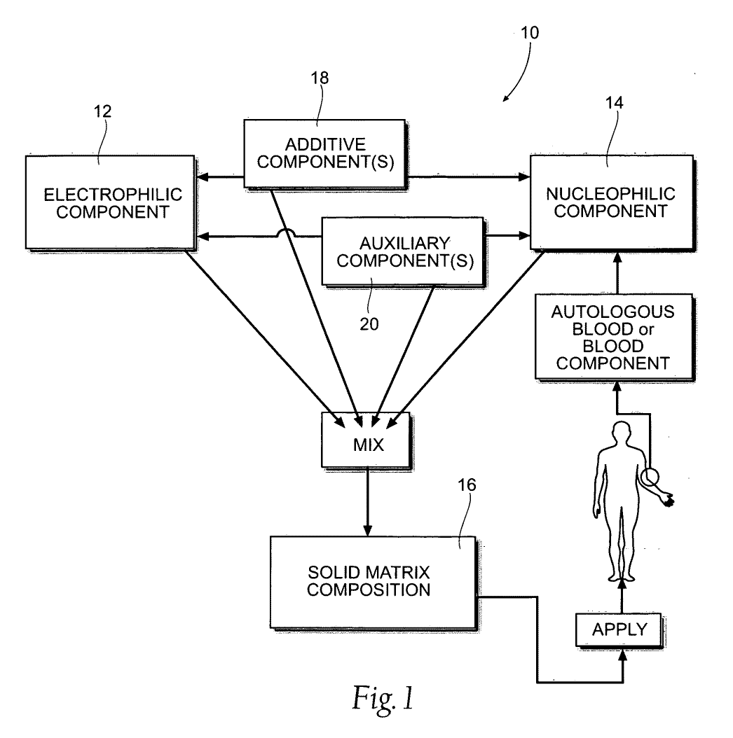

FIG. 1 shows a system 10 for creating families of biocompatible, materials having diverse therapeutic indications. The genus platform for the system 10 includes a biocompatible synthetic electrophilic component 12 mixed with a nucleophilic component 14 that includes a natural, autologous protein. The components 12 and 14 are preferably in solution when mixed, with the base solvent being a water or ethyl alcohol based solvent.

The two components 12 and 14, when mixed in a liquid state, are reactive. When mixed, the two components 12 and 14 react by cross-linking, forming a solid matrix composition 16, or hydrogel. Depending upon the characteristics of the two components 12 and 14 selected, different species of matrix compositions 16 can be formed. These different species lend themselves to use in diverse therapeutic indications.

1. Electrophilic Component

In the illustrated embodiment, the electrophilic component 12 comprises a derivative of a synthetic hydrophilic polymer. The hydrophilic polymers that may be utilized include poly(anhydride esters)(PAE) (available from Polymer Source, Inc. at www.polymersource.com); poly(ethylene glycol) (PEG) (also available from Polymer Source, Inc. at www.polymersource.com), poly(DL-lactides), poly(lactide-co-glycolide (PLA) (available from Birmingham Polymers), poly(ethylene oxide), poly(vinyl alcohol), poly(vinylpyrroldine), poly(ethyloxazoline), and poly(ethylene glycol)-co-poly(propylene glycol) block polymers.

The use of PAE as a hydrophilic electrophilic backbone for a hydrogel will be described in greater detail later.

In alternative embodiments, the hydrophilic polymer can comprise a PEG compound or PEG derivative, a PLA compound, or PLA derivative, or PEG/PLA moieties. In one desired embodiment, the hydrophilic polymer comprises a PEG compound or PEG derivative with a functionality of two or more and a molecular weight in the range of 5000 to 20,000, with a molecular weight of about 10,000 being very desirable.

2. The Nucleophilic Component

In the illustrated embodiment, the nucleophilic component 14 includes a human or animal protein derived from an autologous source. By “autologous source,” it is meant that the human or animal protein is derived from the individual human or animal that is to be treated using the solid matrix composition 16. As will be demonstrated later, the autologous source can include presence of an anticoagulant (e.g., heparin) to facilitate handling.

The autologous protein can be a local region of tissue of the human or animal that is to be treated. Alternatively, or in combination, the autologous protein can be whole blood drawn from the human or animal to be treated, or a blood component or blood derivative that is harvested from blood drawn from the human or animal to be treated. The blood can be drawn at the time that the composition 16 is mixed. Alternatively, the blood can be drawn, processed, and stored beforehand in anticipation of its use in forming the composition 16 during or following later-scheduled surgery or therapeutic procedure (e.g., cosmetic surgery, stem cell delivery, lung resection, etc.).

For example, the blood-derived protein can comprise albumin, or bone marrow stromal stem cells (SSC), or platelet gel (PG), which may be obtained by platelet-rich plasma (PRP) harvested from whole blood. PRP also carries intrinsic growth factors, such as PDGF, TGFb, and FGF. The use of blood or blood compounds derived from autologous blood can itself thus provide intrinsic growth benefits, e.g., the promotion of soft tissue revascularization, and/or acceleration of bone graft healing not otherwise achieved when using pooled, random donor blood products.

Use of a natural, autologous blood or blood compound as the nucleophilic component 14 obviates the use of pooled blood products derived from random human or animal donors. The use of an autologous blood or blood compounds makes possible great compatibility within patients. Such a system could be adapted for human or animal purposes; i.e., human blood would be used for treatment of a human and animal blood would be used when treating an animal.

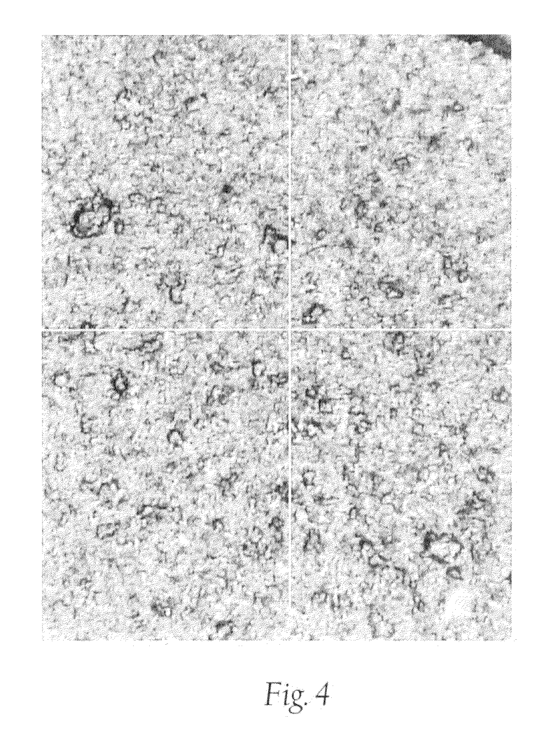

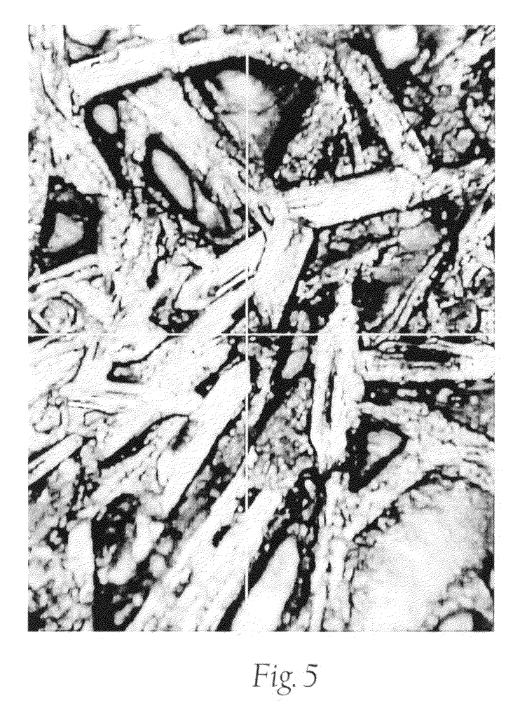

The mixing of an electrophilic material, e.g., a four arm PEG-succinmydil glutarate (PEG-SG), with autologous blood creates a very strong, cross-linked matrix, having a structure and physical characteristics that differ dramatically from those of dry blood. FIG. 4 shows dry human blood at high magnification. Dry human blood is very brittle when handled. FIG. 5 shows, at the same high magnification, a matrix formed by mixing human blood with PEG-SG. The physical cross-linked nature of the structure is very apparent. The presence of PEG-SG has transformed the brittle nature of dry blood into a robust physical structure that can adhere and conform to tissue with beneficial therapeutic results.

3. Matrix Compositions

Species of matrix compositions 16 may be created with a wide range of differentiations. For example, an electrophilic component 12 (e.g., PEG) may be topically applied directly to or injected into a native tissue region, which thereby comprises the nucleophilic component 14. The resulting composition 16 cross-links in situ on or in the native tissue region to provide a desired therapeutic effect, as will be described in greater detail later. This species of composition 16 can be termed a one-component system, i.e., only the electrophilic component 12 need be provided.

As another example, the electrophilic component 12 (e.g., PEG) can be mixed with an autologolous nucleophilic component 14 (e.g., whole blood) at the instant of use. It is this mixture that is topically applied directly to or injected into a native tissue region. The resulting composition 16 cross-links in situ on or in the native tissue region to provide the desired therapeutic effect, as will be described in greater detail later. This species of composition 16 can be termed a two-component system, i.e., the electrophilic component 12 needs to be provided, as does an apparatus (e.g., a syringe) for harvesting the nucleophilic component 14.

As will be described later, kits may be provided to facilate mixing of the electrophilic and nucleophilic components 12 and 14 on site at the instant of use.

4. Additive Components

To promote the cross-linking reaction, additives components 18 (see FIG. 1) may be included to enhance and/or sustain the cross-linking activity between the autologous nucleophilic component 14 and the selected electrophilic component 12.

For example, the additive component 18 can control the reaction pH. Given the known reaction pH range for cross-linking between PEG and a natural protein, the additive component 18 can comprise a buffered base solution (e.g., pH 7.5 to 9.5). In a one-component system, the buffered base solution may be applied or injected into the targeted tissue region prior to, concurrent with, or after the application or injection of the selected electrophilic component 12. As another example, in a two-component system, the buffered base solution may be mixed with selected nucleophilic component 14 (i.e., whole blood) prior to, concurrent with, or after the application or injection of the selected electrophilic component 12.

As another example, the additive component 18 can increase the number of nucleophilic sites to cross-link with the electrophilic component 12. The additive component 18 may include additional human or animal protein, e.g., a human serum albumin (HSA) for human indications, or an animal serum albumin in the case of animal indications. For human applications, the additive component 18 preferably contains less than 20% HSA. The additive component 18 may also include an amine compound, e.g., a poly(ethylene glycol)-amine (PEG-NH₂) compound or lycine.

It should appreciated that the additive component 18 for the nucleophilic compound 14 can include one or more ingredients that affect the activity of the nucleophilic component 14 by various mechanisms, e.g., by controlling reaction pH and/or by increasing the number of functional nucleophilic sites.

The additive components 18 may be added to either the nucleophilic or the electrophilic components 12 and 14, and could also be added to the components 12 and 14 immediately prior to or concurrent with the delivery of the components 12 and 14 to the targeted application site.

5. Auxiliary Components

Based upon the therapeutic indication desired, the solid matrix composition 16 may also incorporate one or more auxiliary components 20 that impart other mechanical and/or therapeutic benefits. These auxiliary components 20 can include fillers, such as glucosamine, glucosaminoglycans, and chondroitin sulfate; anti-inflamatory drugs; rapamycines and analogs, such as everolimus and biolimus or of the kind used on drug-eluting stents by Biosensors International (see. E.g., Prospectus, Biosensors International, Apr. 22, 2005, Registered with the Monetary Authority of Singapore on Apr. 22, 2005); dexamethasone; M-prednisolone; interferon γ-1b; leflunomide; mycophenolic acid; mizoribine; cyclosporine; tranilast; biorest; tacrolimus; taxius; pacitaxel; or taxol; plasticizers, including cellulose and/or non-reactive PEG compounds, such as PEG-hydroxyl compounds; therapeutic agents such as stem cells, antibodies, antimicrobials, collagens, genes, DNA, and other therapeutic agents; hemostatic agents; growth factors; and similar compounds.

The auxiliary components 20 may be added to either the nucleophilic or the electrophilic components 12 and 14, and could also be added to the components 12 and 14 prior to or concurrent with delivery of the components 12 and 14 to the targeted application site.

Autologous blood or an autologous blood compound introduced into a poly(ethylene glycol)-amine (PEG-NH₂) compound, and further combined with a PEG-succinmydil glutarate (PEG-SG), and further including a buffered base solution having, e.g., a pH between 7.5 and 9.5 is a representative example of a composition that will possess positive biological characteristics according to the present invention.

6. Delivery Systems

The components 12, 14, 18, and 20 of the system 10 may be delivered to the targeted application site in several fashions.

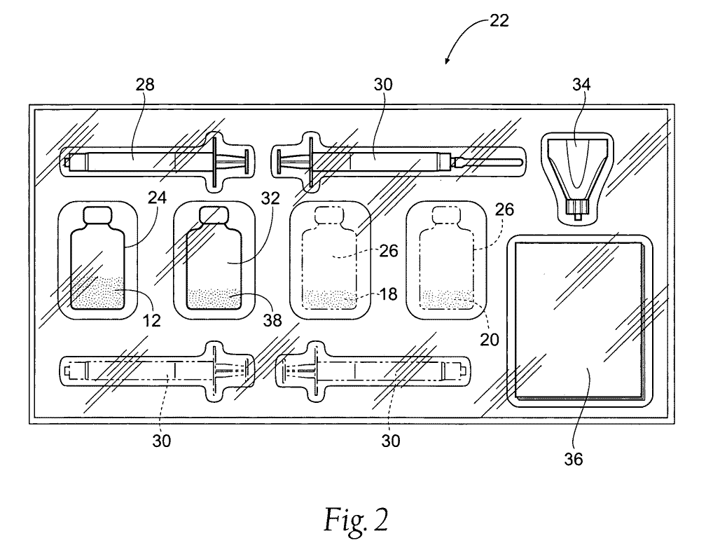

In a preferred embodiment (see FIG. 2), a kit 22 is provided having a vial 24 containing at least a sterile electrophilic component 12 (e.g., a PEG composition). Depending on the compositions specific use, the additives 18 and auxiliary components 20 may also be contained in one or more vials 26 within the kit 22, which are also housed in a sterile fashion. The vials 26 components 18 and 20 may be stored separately from the vial 24 containing PEG composition (as FIG. 2 shows), or in the vial 24 as one mixture with the PEG composition.

The kit 22 may further contain at least one sterile syringe 28 to draw the PEG composition from the vial 24 and deliver the PEG composition to the targeted application site, either topically (e.g., by spraying) or by injection. Further syringes 30 may be included for mixing the PEG composition with additive or auxiliary components, if included. However, it may not be necessary to include a syringe for delivering the PEG composition, for instance in situations where the final composition is to be applied topically. In this instance, the vial 24 could comprise, e.g., a squeeze container or tube from which the ingredients could be expressed by squeezing.

The kit 22 may further contain a syringe 30 or similar device for removing a blood or protein compound from the patient, for instance from a patient's vein, bone marrow, tissue, stem cells, or other area. An empty vial 32 could be provided for storing the blood or blood compound until it is to be mixed with the PEG composition. Further, the kit 22 may include a dual syringe, as known in the art, for mixing together and delivering the blood composition and the PEG composition. The system and method should not be limited by any specific delivery or syringe arrangement, provided that the system would provide means so that the compounds may be mixed together at the delivery site. Processes that provide for a PEG compound to be mixed with a specific patient's blood or blood compound to provide a biologically compatible composition for the above-stated and similar purposes would be considered as falling within the scope of the present invention.

7. Retardant for the Electrophilic Component

It has been discovered that the reactivity of a given nucleophilic component (autologous or otherwise) with a PEG electrophilic component may be controlled other than by pH control by the introduction of a N-hydroxy-succinimide (NHS) compound into the PEG component. Thus, the delivery time of a cross-linked solid matrix composition 16 may be controlled according to specific time schedule. Table 1 compares the relative firmness of protein-PEG based compounds containing differing amounts of NHS.

TABLE 1
Gel Strengths of PEG and NHS Compounds

| Amount of NHS | Average Gel Time | Relative Firmness |
|---|---|---|
| 0% | 7 seconds | Medium |
| 1% | 14 seconds | Medium |
| 5% | 65 seconds | Medium to Soft |
| 10% | 240 seconds | Very Soft |

As shown in Table 1, an increase in the amount of NHS added to the system retards the initial reaction of the system. It should be noted that addition of a predetermined amount of NHS will retard the initial reaction, but after a predetermined time, at or about approximately one (1) hour, all of the gels displayed the same relative firmness.

Accordingly, a nucleophilic component 14 comprising autologous blood or an autologous blood compound can be combined with a free NHS compound (which would act as a retardant) and could be injected as a cross-linked product. This composition 16 can be integrated with a bandage, gel foam, or other topical product to deliver biological materials according to the present invention.

B. Biodegradability and Biocompatibility

Three PEG-based hydrogel compositions were formulated and injected into the back tissue of a living rat host.

Composition 1 comprised a hydrogel material that included a non-autologous protein component. The electrophilic component comprised a multifunctional PEG-succinimidyl glutarate compound, such as PEG-tetra-succinimidyl glutarate. As a shorthand reference, these compounds will be referred to as PEG-SG. The multifunctional four-arm PEG-SG (250 mg) (10,000 m/w) was mixed with sterile water (1.5 ml) to yield a PEG-SG concentration of 166 to 170 milligrams/ml. The nucleophilic component comprised 25% HSA (Bayer) (3 ml) mixed with sterile water (1.9 ml) to yield 15% HSA Solution (HSA density of 1.07 g/cc, and a pH of about 8.5). The PEG-SG component (1 ml) and the 15% HSA component (1 ml) were mixed though a static mixer and injected in equal aliquoits (0.5 ml each) into first and second back tissue sites of the rat. Composition 1 served as a control.

Composition 2 comprised a hydrogel material that included an autologous protein component comprising anticoagulated (using heparin) whole blood drawn from the host rat. The electrophilic component comprised the multifunctional four-arm PEG-SG (10,000 m/w) used for Component 1, but formulated at a higher concentration. The electrophilic component comprised PEG-SG (250 mg) mixed with sterile water (0.5 ml), yielding a PEG-SG concentration of 500 milligrams/ml. The nucleophilic component comprised heparinized autologous whole blood of the rat (1 ml) (anticoagulant ratio: 1 ml heparin to 5 ml whole blood). Additives were mixed with the nucleophilic component; namely, a base buffer solution of tris-hydroxymethylaminomethane (Tris) (400 mg), and an amine compound—multifunctional four-arm poly(ethylene glycol)-amine (PEG-NH₂) (50 mg)—to increase the number of nucleophilic sites to cross-link with the electrophilic component. The PEG-SG component (0.5 ml) and the autologous blood component (with additives) (0.5 ml) were mixed though a static mixer and injected into a third back tissue sites of the rat.

Composition 3, like Composition 2 comprised a hydrogel material that included an autologous protein component comprising anticoagulated (heparinized) whole blood drawn from the host rat. The electrophilic component comprised the same multifunctional four-arm PEG-SG (10,000 m/w) used for Component 3, formulated at the same concentration—i.e., PEG-SG (250 mg) mixed with sterile water (0.5 ml), yielding a PEG-SG concentration of 500 milligrams/ml. The nucleophilic component comprised the same amount of heparinized autologous whole blood of the rat used for Component 2—i.e., whole blood (1 ml) (anticoagulant ratio: 1 ml heparin to 5 ml whole blood). Additives were mixed with the nucleophilic component, but in different amounts than in Component 2; namely, a base buffer solution of tris-hydroxymethylaminomethane (Tris) (500 mg), and an amine compound-multifunctional four-arm poly(ethylene glycol)-amine (PEG-NH₂) (180 mg). Component 3 therefore had a higher concentration of nucleophilic sites than Component 2. The PEG-SG component (0.5 ml) and the autologous blood component (with additives) (0.5 ml) were mixed though a static mixer and injected into a fourth back tissue sites of the rat.

The hydrogel materials gelled within the tissue sites and resided there for thirty days. After thirty days, the materials had all degraded by hydrolysis to various degrees. Composition 3 had entirely degraded. Composition 2 had degraded, but to a lesser extent, with a small amount of material still present. Composition 1 had also degraded, but to a lesser extent than Composition 2, with a larger amount of material still remaining.

In tissue contiguous to all three Compositions, there was no visual indication of inflammatory reactions. Skin tissue from tissue contiguous to Composition 2 was processed for routine histology preparation and stained with hematoxylin and eosin. Microscopic evaluation of the tissue was not indicative of an inflammatory reaction.

### II. Bio-Erodable Compositions

A. System Overview

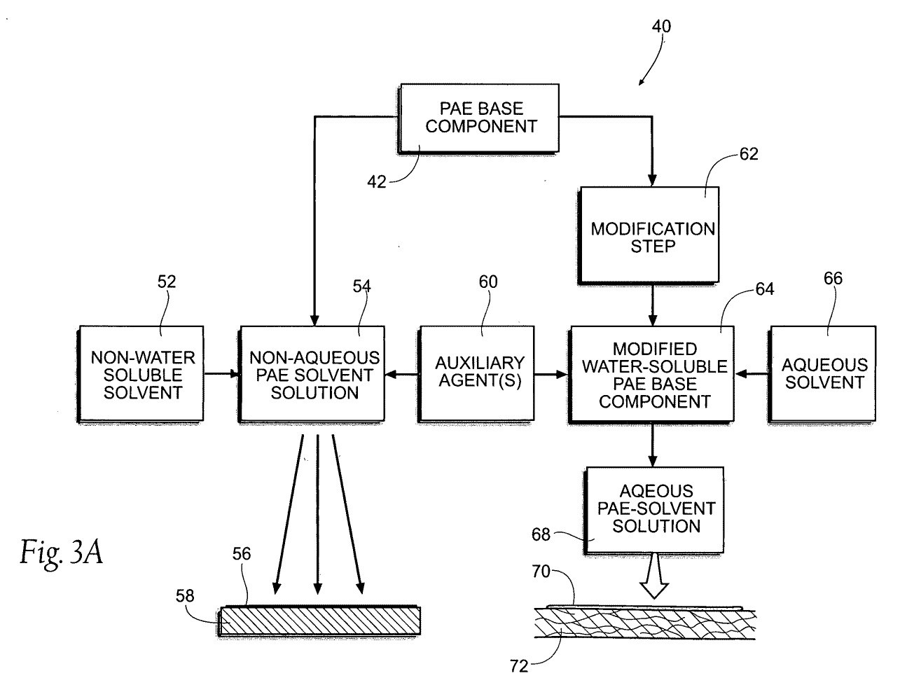

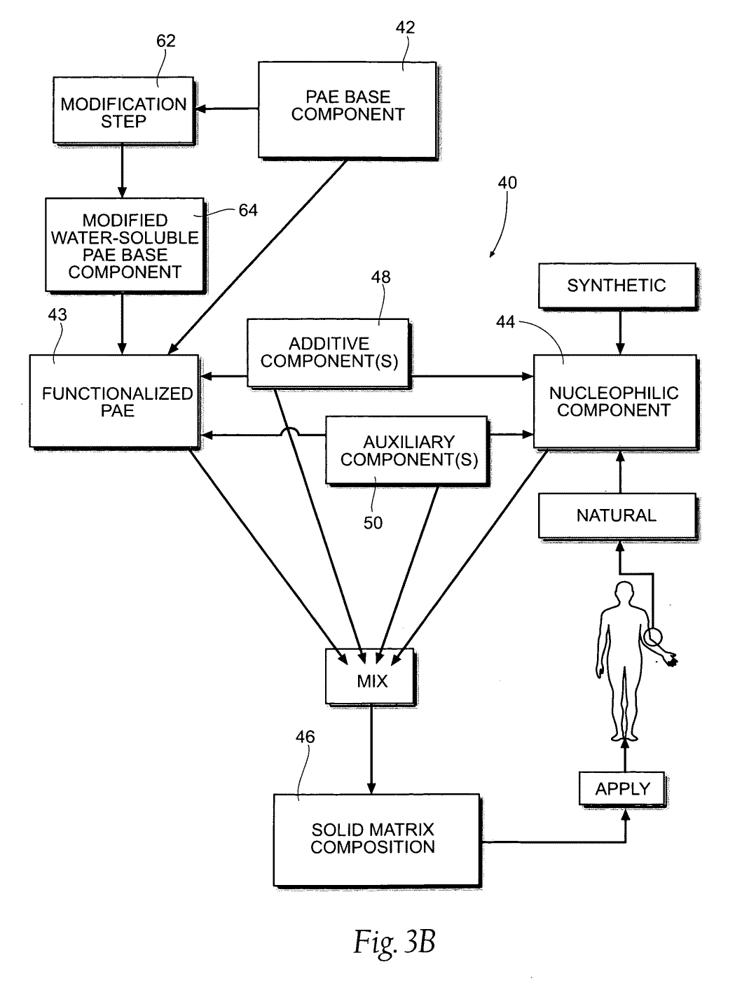

FIG. 3A shows a system 40 for creating families of biocompatible, bio-erodable materials having diverse therapeutic indications. The genus platform for the system 40 includes a biocompatible component 42 comprising a poly(anhydride ester) (PAE) material.

The poly-anhydride component 42 comprises an aromatic poly(anhydride ester) that can be characterized by possessing a repeating unit with the basic backbone structure:

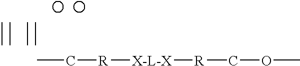

wherein L is a linking group, and each R and X is independently selected to provide aromatic poly-anhydrides that hydrolyze to form a salicylic acid or salicyclic acid derivative. Examples of appropriate salicylates include, but are not limited to, diflunisal, diflucan, thymotic acid, 4,4-sulfinyldinailine, 4-sulfanilamidosalicyclic acid, sulfanilic acid, sulfanilylbenzylamine, sulfaloxic acid, succisulfone, salicylsulfuric acid, salsallate, salicyclic alcohol, salicyclic acid, succisulfone, salicysulfuric acid, salsallate, salicylic alcohol, salicylic acid, orthocaine, mesalamine, gentisic acid, enfenamic acid, cresotic acid, aminosalicylic acid, aminophenylacetic acid, acetyisalicylic acid, and the like.

In a desired embodiment, the active agent is salicylic acid. Salicylates have been used routinely as anti-inflammatory, antipyretic, analgesic, and anti-oxidant agents. That poly (anhydride esters) based upon salicylic acid are biocompatible is accepted, as is the ability to administer such compositions to an animal through a variety of routes, such as orally, subcutaneously, intramuscularly, intradermally and topically.

Further details of base PAE compounds that can serve as the genus PAE platform according to the present invention are disclosed in International Publication Number WO 2004/045549, and U.S. patent application Ser. No. 10/861,881, filed Jun. 4, 2004 (Publication No. US 2005/0048121), which are incorporated herein by reference.

PAE component 42 can be synthesized in various ways. In one representative embodiment, a poly(anhydride ester) (PAE) is prepared, as follows:

### Example 1

### Poly(Anhydride Ester) (PAE) Synthesis

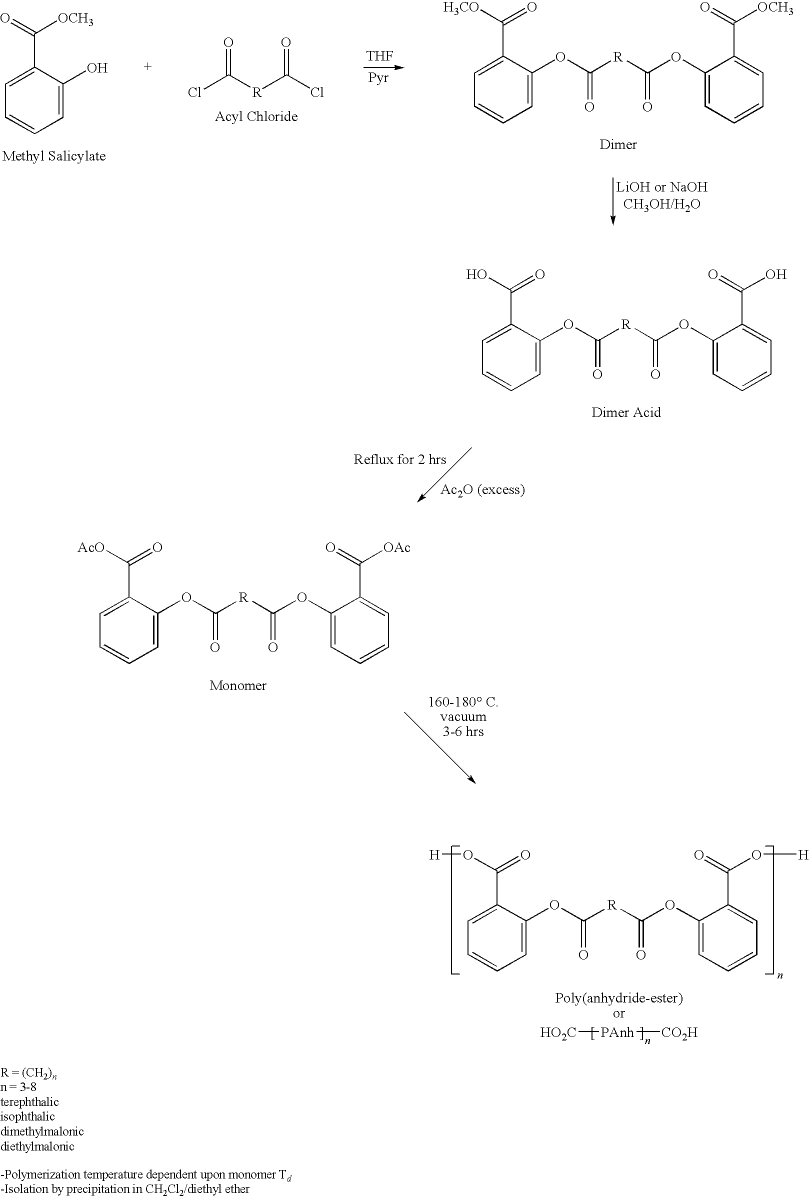

Once synthesized, the genus PAE platform can be further formulated in various ways to perform diverse therapeutic functions, as shown in FIGS. 3A and 3B.

B. Non-Water Soluable, Biocompatible and Bio-Erodable PAE Compositions

The genus PAE component 42 synthesized according to Example 1 is not water soluable. For use (see FIG. 3A), the genus PAE component 42 can be placed into solution by mixing with a non-water-based solvent 52, e.g., acetone or methylene chloride, or TCE, to form a non-aqueous PAE-solvent solution 54. The non-aqueous PAE-solvent solution 54 can be applied, e.g., by spraying, dipping, or painting, to the surface of synthetic biocompatible material as a coating 56. The synthetic material can comprise plastic, or metal, or fabric, or ceramic. The synthetic material can be formed, e.g., into a prosthesis or like device 58 intended for implantation in an animal body. The formed device 58 can comprise, e.g., an orthopedic prosthesis to replace or augment bone, or a valve prosthesis to replace or augment a heart valve, or a stent or vascular graft.

When the PAE component 42 includes salicylic acid as a base agent, the PAE-solvent coating 56 provides a protective, anti-inflammatory effect to impart improved comfort, tolerance, healing, and bio-acceptance to the implanted device 58 in a recipient.

Alternatively, or in combination with a salicylic acid base agent, the non-aqueous PAE-solvent solution can additionally incorporate other selected auxiliary agents 60 having other desired therapeutic effects, and/or effects that enhance the anti-inflammatory effect, to impart improved comfort, tolerance, and bio-acceptance to the implanted device 58 in the recipient. Such agents 60 can comprise, e.g., anti-inflamatory drugs; rapamycines and analogs, such as everolimus and biolimus; dexamethasone, or of the kind used on drug-eluting stents by Biosensors International (see. E.g., Prospectus, Biosensors International, Apr. 22, 2005, Registered with the Monetary Authority of Singapore on Apr. 22, 2005); M-prednisolone; interferon γ-1b; leflunomide; mycophenolic acid; mizoribine; cyclosporine; tranilast; biorest; tacrolimus; taxius; pacitaxel; or taxol; botox; lydicane; Retin A Compound; glucosamine; chondroitin sulfate; or Geldanamycin analogs 17-AAG or 17-DMAG (obtained from Kosan Biosciences, Hayward, Calif.).

Once applied to the implantable device 58, the PAE-solvent coating 56 will over time biodegrade and/or bioerode via native hydrolysis and enzymatic mechanisms within the body of the patient. Because the breakdown products of PAE include aspirin and other agents that are themselves therapeutic, the PAE-solvent coating 56 can be used to reduce pain, reduce inflammation, reduce scarring, promote wound healing, reduce topical pain, reduce biofilm (i.e., infection), and provide an antiseptic effect.

C. Water Soluable, Biocompatible and Bio-Erodable PAE Compositions

As FIG. 3A also shows, the genus PAE component 42 synthesized according to Example 1 can be modified in a modification step 62 to form a modified PAE base component 64 that is water soluable. The modified water soluable base component 64 can be placed into solution by mixing with an aqueous solvent 66, e.g., sterile water, to form an aqueous PAE-solvent solution 68. The aqueous PAE-solvent solution 68 can be formulated into a cream or topical dressing 70 that can be applied in conventional fashion upon a skin surface 72, e.g., at a site of localized infection, a wound site, or a burn site.

The modification step 62, altering the genus PAE component 42 to form the modified water-soluable PAE base component 64 can be accomplished in various ways. For example, the modification step 62 can comprise replacing the “R” group in Example 1 with a PEG group:

wherein the PEG group comprises —(CH₂ CH₂—O—)ₙ—; and

wherein n≧1, with increasing values of n leading to a greater degree of water soluability.

When the PAE base component 42 includes salicylic acid as a base agent, the ultimate aqueous PAE-solvent solution 68, when applied topically as a cream 70 or incorporated into a wound dressing, can provide a protective effect to moderate inflammation and/or impart improved comfort, protection against infection, and healing at the application site.

Alternatively, or in combination with a salicylic acid base agent, the aqueous PAE-solvent solution 68 can additionally incorporate other selected agents 60 having other effects and/or an effect that enhances the desired therapeutic effect at the application site. As previously described, such auxiliary agents 60 can comprise, e.g., anti-inflamatory drugs; rapamycines and analogs, such as everolimus and biolimus, or of the kind used on drug-eluting stents by Biosensors International (see. E.g., Prospectus, Biosensors International, Apr. 22, 2005, Registered with the Monetary Authority of Singapore on Apr. 22, 2005); dexamethasone; M-prednisolone; interferon γ-1b; leflunomide; mycophenolic acid; mizoribine; cyclosporine; tranilast; biorest; tacrolimus; taxius; pacitaxel; or taxol; botox; lydicane; Retin A Compound; glucosamine; chondroitin sulfate; or Geldanamycin analogs 17-AAG or 17-DMAG (obtained from Kosan Biosciences, Hayward, Calif.).

Once applied to the application site, the aqueous PAE-solvent solution 68 will over time biodegrade and/or bioerode via native hydrolysis and enzymatic mechanisms.

Because the breakdown products of PAE include aspirin and other agents that are themselves therapeutic, the aqueous PAE-solvent solution 68 can be used to reduce pain, reduce inflammation, reduce scarring, promote wound healing, reduce topical pain, reduce biofilm (i.e., infection), and provide an antiseptic effect at the application site.

D. Functionalized Biocompatible Bio-Erodable PAE Compositions

In another embodiment (see FIG. 3B), the PAE genus component 42 can be functionalized to comprise a biocompatible electrophilic component 43 comprising PAE. By “functionalized electrophilic component comprising PAE” it is meant that the basic molecular segment or backbone of PAE is modified to generate or introduce a new reactive electrophilic functional group (e.g., a succinimidyl group) that is capable of undergoing reaction with another functional nucleophilic group (e.g., a thiol or an amine group) to form a covalent bond. The functionalized electrophilic poly(anhydride ester) component 43 is mixed with a selected nucleophilic component 44. The components 43 and 44 are preferably in solution when mixed. The two components 43 and 44, when mixed in a liquid state, are reactive. When mixed, the two components 43 and 44 react by cross-linking, forming a solid matrix composition 46, or hydrogel. Depending upon the characteristics of the two components 43 and 44 selected, different species of matrix compositions 46 can be formed. These different species lend themselves to use in diverse therapeutic indications.

### 1. Electrophilic Component

In the illustrated embodiment, the electrophilic component 43 comprises a base poly(anhydride ester) (PAE) component 42 that has been electrophilically derivatized (“functionalized”) with a functionality of at least one.

As before described, the poly-anhydride component 42 comprises an aromatic poly(anhydride ester) that can be characterized by possessing a repeating unit with the basic backbone structure

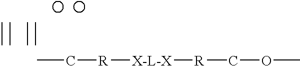

wherein L is a linking group, and each R and X is independently selected to provide aromatic poly-anhydrides that hydrolyze to form a salicylic acid or salicyclic acid derivative. Examples of appropriate salicylates include, but are not limited to, diflunisal, diflucan, thymotic acid, 4,4-sulfinyldinailine, 4-sulfanilamidosalicyclic acid, sulfanilic acid, sulfanilylbenzylamine, sulfaloxic acid, succisulfone, salicylsulfuric acid, salsallate, salicyclic alcohol, salicyclic acid, succisulfone, salicysulfuric acid, salsallate, salicylic alcohol, salicylic acid, orthocaine, mesalamine, gentisic acid, enfenamic-acid, cresotic acid, aminosalicylic acid, aminophenylacetic acid, acetyisalicylic acid, and the like.

In a desired embodiment, the active agent is salicylic acid due to its desirable anti-inflammatory, antipyretic, analgesic, and anti-oxidant effects. The ability to functionalize such compounds and to cross-link them in situ into hydrogel structures has not heretofore been contemplated or appreciated.

Further details of base PAE compounds that can be functionalized according to the present invention are disclosed in International Publication Number WO 2004/045549, and U.S. patent application Ser. No. 10/861,881, filed Jun. 4, 2004 (Publication No. US 2005/0048121), which are incorporated herein by reference.

PAE can be synthesized in various ways. In one representative embodiment, a poly(anhydride ester) (PAE) is prepared according to preceding Example 1.

The poly(anhydride ester) (PAE) is thereafter derivatized (i.e., functionalized) to include electrophilic function groups. The following reaction Examples 2 and 3, illustrate two methods of functionalization of polyanhydride esters.

### Example 2

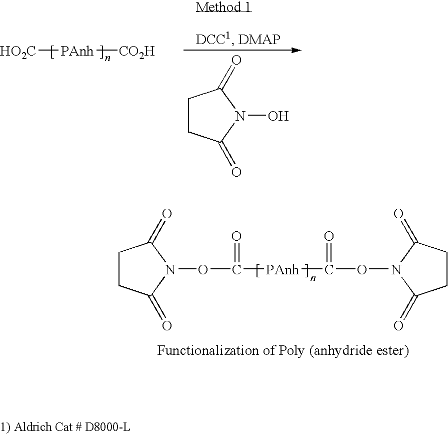

### Example 3

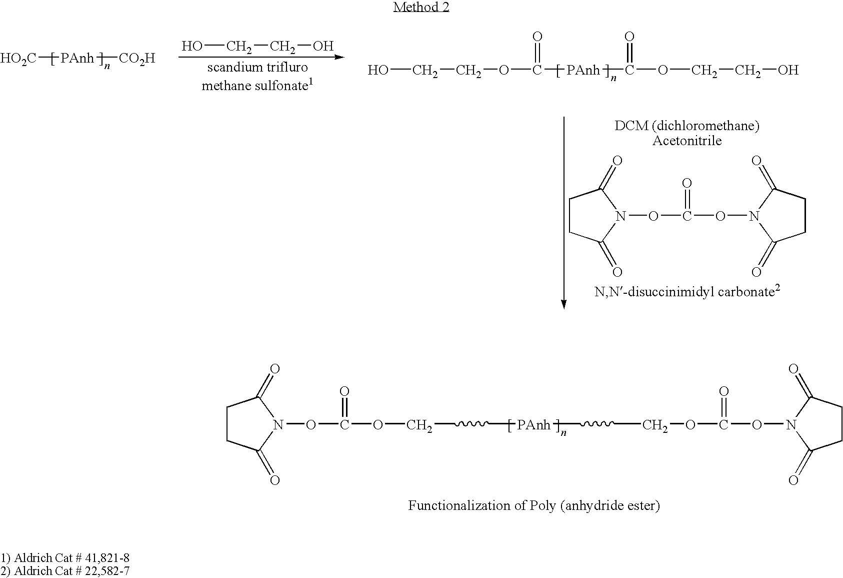

The resultant functionalized electrophilic PAE backbone can be linear (single functional or bi-functional) or branched (multifunctional). Multifunctional branches can be added to a single functional group, to impart multifunctionality.

The functionalized component 43 can be either non-water soluable or water soluable.

If the PAE base component 42 is not modified by a modification step 62 prior to being functionalized, the functionalized component 43 remains non-water soluable, and would pose difficulties if it is desirable to react the component 42 with other components that are soluable in water, for example, protein.

Conversely, if the PAE base component 42 is modified by a modification step 62 (of the kind previously described) prior to being functionalized, the functionalized component 43 will be water soluable and will readily react with other components, like protein, that are soluable in water.

The form of the functionalized component 43 can be selected according to the intended therapeutic indication.

When the therapeutic indication involves the application of the mixture of functionalized components 43 and 44 as a biocompatible and bio-erodable coating on a prosthetic surface, water soluability may not be a necessary and/or desirable attribute. Thus, the functionalized component 43 need not be water soluable. In this circumstance, the base solvent can comprise acetone, methylene chloride, or TCE, as previously described, and the nucleophilic component 44 can be selected among non-water soluable materials.

When the therapeutic indication involves the application, spraying, or injection of the functionalized electrophilic PAE component 43 into or on to human or animal tissue, with the expectation that the electrophilic PAE component 43 will cross-link with a native, water soluable nucleophilic amine group (e.g. blood), the functionalized component 43 is desirably made water soluable (by prior modification in step 62 of the base PAE component 42, as previously described). In this circumstance, the base solvent desirably comprises a water-based solvent.

Alternatively, it is believed that a non-water soluable functionalized electrophilic PAE component 43 can be placed into solution with an ethyl alcohol based solvent and will cross-link with a native, water soluable nucleophilic amine group (e.g. blood).

The resultant functionalized electrophilic PAE backbone shown above is not soluable in water, but is soluable in solvents such as acetone, methylene chloride, or TCE. The resulting polymer are desirably cross-liked in the presence of the solvent with nucleophilic materials that are not water soluable—i.e., synthetic nucleophilic materials—to form a hydrogel that degrades, at least in part, by a surface erosion process, and not solely by liquification by hydrolysis.

If cross-linking with a water soluable nucleophilic material (e.g., a hydrophilic protein like blood or derivatives thereof) is desired, the base PAE component 42 is desirably modified by a modification step 62 to be made water-soluable, e.g., by replacing the “R” group in Example 1 with a PEG group

wherein the PEG group comprises —(CH₂ CH₂—O—)ₙ—; and

wherein n≧1, with increasing values of n leading to greater degrees of water soluability.

The modified, water soluable PAE component 64 can thereafter be derivatized (i.e., functionalized) to include electrophilic function groups in manner illustrated in Examples 2 or 3.

The resulting water soluable PAE component 64 can be cross-liked in the presence of a water-based solvent with either synthetic or naturally occurring water soluable nucleophilic materials to form a hydrogel that degrades in situ, at least in part, by a surface erosion process, and not solely by liquification by hydrolysis.

Because the breakdown products of PAE (whether water soluable or not water soluable) include aspirin and other agents that are themselves therapeutic, hydrogels based upon functionalized PAE can be used to reduce pain, reduce inflammation, reduce scarring, promote wound healing, reduce topical pain, coat stents and vascular grafts, reduce biofilm (i.e., infection), and provide an antiseptic effect.

### 2. The Nucleophilic Component

The nucleophilic component 44 includes a material with nucleophilic groups, e.g., amines, or thiols. The component 44 can comprise a synthetic material, e.g. a poly(ethylene glycol)-amine (PEG-NH₂) compound, lycine, or a functionalized nucleophilic poly(anhydride ester). Alternatively, or in combination, the component 44 can comprise a naturally occurring nucleophilic material. For example, the nucleophilic component 44 can include a hydrophilic protein or derivatives thereof, such as serum, serum fractions, blood, and a blood component, as well as solutions of albumin, gelatin, antibodies, fibrinogen, and serum proteins, as well as collagen, elastin, chitosan, and hyaluronic acid. The protein structure may be derived from non-autologous (i.e., pooled) sources, or from autologous sources, as described above. Further, the protein structure need not be restricted to those found in nature. An amino acid sequence can be synthetically designed to achieve a particular structure and/or function and then incorporated into the nucleophilic component 44. The protein can be recombinantly produced or collected from naturally occurring sources.

As previously described, to promote the cross-linking reaction, one or more additives components 48 may be included to enhance and/or sustain the cross-linking activity between the nucleophilic component 44 and the selected electrophilic component 43. The additive component 48 can comprise a buffering solution to affect the pH of the cross-linking reaction. Alternatively, or in combination, the additive component 48 can comprise a material that increases the number of nucleophilic sites available for cross-linking with the electrophilic component 43. The additive component 48 may include a N-hydroxy-succinimide (NHS) compound to retard the rate of the cross-linking reaction, as previously described.

As also previously described, the solid matrix composition 46 may also incorporate one or more auxiliary components 50 that impart other mechanical and/or therapeutic benefits. These auxiliary components 50 can include fillers, such as glucosamine, glucosaminoglycans, and chondroitin sulfate; anti-inflamatory drugs; rapamycines and analogs, such as everolimus and biolimus or of the kind used on drug-eluting stents by Biosensors International (see. E.g., Prospectus, Biosensors International, Apr. 22, 2005, Registered with the Monetary Authority of Singapore, on Apr. 22, 2005); dexamethasone; M-prednisolone; interferon γ-1b; leflunomide; mycophenolic acid; mizoribine; cyclosporine; tranilast; biorest; tacrolimus; taxius; pacitaxel; or taxol; Geldanamycin analogs 17-AAG or 17-DMAG (obtained from Kosan Biosciences, Hayward, Calif.); plasticizers, including cellulose and/or non-reactive PEG compounds, such as PEG-hydroxyl compounds; therapeutic agents such as stem cells, antibodies, antimicrobials, collagens, genes, DNA, and other therapeutic agents; hemostatic agents; growth factors; and similar compounds.

The auxiliary components 50 may be added to either the nucleophilic or the electrophilic components 43 and 44, and could also be added to the components 43 and 44 prior to or concurrent with delivery of the components 42 and 44 to the targeted application site.

The composition 46 may be delivered using the kit shown in FIG. 2. The electrophilic PAE component 43 would be contained in the vial 24.

### III. Therapeutic Indications

A. Collagen Restoration/Replacement

A composition 16 comprising a biocompatible synthetic electrophilic component 12 mixed with a nucleophilic component 14 that includes a natural, autologous protein—or a composition 46 comprising functionalized electrophilic poly-anhydride component 43 mixed with a nucleophilic component 44 (autologous or otherwise) can be applied topically or by injection for the restoration or replacement of collagen. This indication includes augmenting soft tissue in humans or animals, as well as cosmetic applications.

For example, the composition 16 or 46 may be injected as a void filling composition. It also may be placed into body cavities, with or without collagen, for example a nasal airway, or an organ of the gastro-intestinal track, to arrest localized bleeding and/or promote healing following trauma, injury, or surgery. Alternatively, the composition 16 may be applied as a topical cosmetic or therapeutic composition, used, e.g., in connection with creams, shampoos, soaps, and oils, for dermatological, cleansing, or similar purposes. The composition 16 or 46 can include, with or without collagen, auxiliary components such as rapamycine or analogs like everolimus or biolimus, which can promote a reduction of scaring after plastic surgery performed on the face, body, or other external skin area. Conjugates in the composition 16 or 46 can be absorbed in or on the surface of the skin or hair and may assist in possible replenishment of skin or hair structure, as well as possible healing of tissue, muscle, and bones.

In this indication, the nucleophilic component 14 may be derived from human tissue with or without a buffer solution, human blood or a human blood component with or without a buffer solution, and optionally with a protein, e.g., human serum albumin (HSA). The electrophilic component 12 may be a PEG-succinimidyl glutarate compound, such as PEG-tetra-succinimidyl glutarate (PEG-SG), or a functionalized poly-anhydride compound. Further additives, such as glucosamine, chondroitin sulfate, and lydicane may be added to the composition.

As an example of the effectiveness of the composition 16 based upon PEG-SG, cross-linked polymers were prepared with albumin solutions consisting of differing percentages of HSA concentration. The albumin solutions were mixed with a PEG-SG composition, and allowed to gel for a specified time. The compounds 16 were allowed to set for five (5) minutes, and the hardness of the compounds was noted. The results were recorded in Table 2.

TABLE 2
Gel Formation and Strengths of PEG-SG Compositions

| Gel Time | % Human serum | Firmness (after 5 |
|---|---|---|
| (seconds) | albumin (HSA) | minutes) |
| 10 | 25 | Medium |
| 15 | 20 | Medium/Soft |
| 25 | 15 | Soft |

As Table 2 indicates, hardness of the composition increases with the percentage of HSA, or, conversely, the flexibility of the compound increases and brittleness of the composition is reduced as the HSA concentration is reduced. The lower percentages result in a superior product. Likewise, the product can replace the use of bovine-based collagen products previously used.

It was determined the firmness of the composition also changes when the pH of the buffered HSA composition is altered. Table 3 shows the relative firmness of a gel formed from a buffered HSA combined with a PEG composition. Generally, as the pH increases, so does firmness of the compounds.

TABLE 3
Buffered Human Serum Albumin/PEG Gel Formations

| pH | Average Gel Time | Relative Firmness |
|---|---|---|
| 9.70 | <3 seconds | Hard |
| 9.50 | <3 seconds | Medium-Hard |
| 9.30 | <3 seconds | Medium-Hard |
| 9.00 | 4.1 seconds | Medium-Hard |
| 8.80 | 6.0 seconds | Medium |
| 8.60 | 11.9 seconds | Medium |
| 8.50 | 14.8 seconds | Medium |
| 8.20 | 64.6 seconds | Soft |

B. Drug Delivery

A composition 16 comprising a biocompatible synthetic electrophilic component 12 mixed with a nucleophilic component 14 that includes a natural, autologous protein or a composition 46 comprising functionalized electrophilic poly-anhydride component 43 mixed with a nucleophilic component 44 (autologous or otherwise)—can be used for drug delivery systems. In this indication, the composition 16 or 46 may be used as a carrier for a biologically active-material delivered to a patient. The composition 16 or 46 including the biologically active material may be formed in situ or as a preformed implant. The biologically active material could be covalently bound to the cross-linked composition 16 or 46 and be released as the result of the degradation of the cross-linked composition 16 or the bio-erosion of the cross-linked composition 46. Likewise, the biologically active material could be released through a diffusion process.

An example of a drug delivery composition includes blood or a blood component, alternatively with a protein compound (such as HSA), combined with a PEG compound, preferably a PEG-SG compound. A drug delivery composition may also comprise a protein compound combined with a functionalized poly-anhydride material. Additives, such as glucosamine, chondroitin sulfate, stem cells, botox, lydicane, Retin A® Compound, rapamicine, compositions of the kind used on drug-eluting stents' by Biosensors International (see. E.g., Prospectus, Biosensors International, Apr. 22, 2005, Registered with the Monetary Authority of Singapore on Apr. 22, 2005); dexamethasone, everolimus, sirolimus, tacrolimus, taxius, or other additives previously mentioned, could be placed in the drug delivery system and injected in targeted areas of the body. For example, the composition 16 or 46 carrying autologous growth factors and/or stem cells (mesenchymal progenitor cells) is well suited for injection in liquid form into an intervertebral disc space. Upon gelation, the composition 16 or 46 will begin to slowly release these materials to treat degeneration of the disc (i.e., to regenerate the disc).

A drug delivery system incorporating the composition 16 or 46 incorporating an autologous protein is advantageous over previous delivery systems. Because the nucleophilic compound is provided from an autologous blood base, specifically from the individual patient, concerns of impurity and contamination of the blood source are reduced. Thus, the delivery, system incorporating the composition 16 or 46 is more conducive for patients who may be at risk from receiving blood that their immune systems may reject, such as AIDS patients or anemic patients. The presence of the hydrogel keeps the drug or other additive (e.g., stem cells) localized, so they are not immediately disbursed away from the intended treatment site. As a result, a higher concentration of the drug or additive remains at the intended treatment site for a longer period of time. Furthermore, the presence of an autologous blood or blood component in the hydrogel provides a more natural environment for an additive such as stem cells, which itself comprises a blood-based material.

C. Sealants and Adhesives

A composition 16 comprising a biocompatible synthetic electrophilic component 12 mixed with a nucleophilic component 14 that includes a natural, autologous protein—or a composition 46 comprising functionalized electrophilic poly-anhydride component 43 mixed with a nucleophilic component 44 (autologous or otherwise)—can be used as a tissue sealant, or adhesive, or a hemostatic device. The composition 16 or 46 can be applied to tissue or organs, such as lungs, abdominal areas, vascular tissue, gastrointestinal tissue, or any other tissues, to stop the leakage of air, blood or other fluid through an incision or anastomoses.

D. Surgical Adhesions

A composition 16 comprising a biocompatible synthetic electrophilic component 12 mixed with a nucleophilic component 14 that includes a natural, autologous protein—or a composition 46 comprising functionalized electrophilic poly-anhydride component 43 mixed with a nucleophilic component 44 (autologous or otherwise)—can be used to assist in reducing the formation of adhesions after surgery. The composition 16 or 46 can include auxiliary components such as rapamycine or analogs like everolimus or biolimus, which can enhance the adhesion reduction effect following surgery. The composition 16 can be applied to a damaged tissue or organ, with the composition providing a protective hydrogel coating on the damaged area. As previously stated, the use of an autologous blood source for the nucleophilic component of the composition 16 or 46 further reduces complications in applying a foreign material to certain high-risk patients.

E. Other Indications

A composition 16 comprising a biocompatible synthetic electrophilic component 12 mixed with a nucleophilic component 14 that includes a natural, autologous protein—or a composition 46 comprising functionalized electrophilic poly-anhydride component 43 mixed with a nucleophilic component 44 (autologous or otherwise)—can be used as an embolic material. The composition 16 can be formulated to biodegrade or erode slowly, while the clotting process progresses. For example, the composition 16 can comprise a transcatheter embolic material for clotting intracranial (or extracranial) aneurysms, or arterial venous malformations (AVM).

A composition 16 comprising a biocompatible synthetic electrophilic component 12 mixed with a nucleophilic component 14 that includes a natural, autologous protein—or a composition 46 comprising functionalized electrophilic poly-anhydride component 43 mixed with a nucleophilic component 44 (autologous or otherwise)—can be injected into cardial tissue to treat arrythmias. The composition 16 would be injected instead of, e.g., forming an intracardia lesion by the application of radio frequency energy, to serve to interrupt aberrant conduction pathways.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

I claim:

1. A hydrogel composition for application to a tissue region of an animal comprising
a first component comprising an electrophilic polymer material, and
a second component comprising a nucleophilic material comprising autologous blood or an autologous blood component obtained from the animal that, when mixed in solution with the first component and applied to the tissue region, cross-links in situ with the first component to form a non-liquid structure.

2. A hydrogel composition according to claim 1
wherein the first component includes poly(ethylene glycol) (PEG), or poly(DL-lactides), or poly(lactide-co-glycolide (PLA), or poly(ethylene oxide), or poly(vinyl alcohol), or poly(vinylpyrroldine), or poly(ethyloxazoline), or poly(ethylene glycol)-co-poly(propylene glycol) block polymers-, or combinations thereof.

3. A hydrogel composition according to claim 1
wherein the first component includes a functionalized electrophilic poly(anhydride ester) material or a functionalized electrophilic derivative of a poly(anhydride ester) material.

4. A hydrogel composition according to claim 1
wherein the second component includes a blood anticoagulant.

5. A hydrogel composition according to claim 4
wherein the blood anticoagulant includes heparin.

6. A hydrogel composition for application to a tissue region of an animal comprising
a first component comprising a functionalized electrophilic poly(anhydride ester) material or an functionalized electrophilic derivative of a poly(anhydride ester) material, and
a second nucleophilic component that, when mixed in solution with the first component and applied to the animal tissue region, cross-links in situ with the first component to form a non-liquid structure.

7. A hydrogel composition according to claim 6
wherein the second component comprises autologous blood or an autologous blood component obtained from the animal.

8. A hydrogel composition according to claim 7 wherein the second component includes a blood anticoagulant.

9. A hydrogel composition according to claim 8
wherein the blood anticoagulant includes heparin.

10. A hydrogel composition according to claim 1 or 6
further including an additive component comprising a buffer solution, or a component that increases the number of nucleophilic sites, or a drug agent, or a therapeutic agent, or a filler, or a plasticizer, or a hemostatic agent, or combinations thereof.

11. A hydrogel composition according to claim 10
wherein the therapeutic agent includes stem cells, or antibodies, or antimicrobials, or collagen, or a gene, or DNA, or combinations thereof.

12. A hydrogel composition according to claim 1 or 6
further including a therapeutic agent comprising an anti-inflamatory drug; rapamycine and analogs, such as everolimus and biolimus; dexamethasone; M-prednisolone; interferon γ-1b; leflunomide; mycophenolic acid; mizoribine; cyclosporine; tranilast; biorest; tacrolimus; taxius; pacitaxel; or taxol; botox; lydicane; Retin A Compound; glucosamine; chondroitin sulfate; or Geldanamycin analogs 17-AAG or 17-DMAG.

13. A method of treating an animal comprising
providing a hydrogel composition as defined in claim 1 or 6, and
applying the hydrogel composition to a tissue region of the animal.

14. A method according to claim 12
wherein the hydrogel composition is applied to fill a tissue void, or to deliver a drug, or to deliver a therapeutic agent, or to seal tissue, or as a tissue adhesive, or as an hemostatic agent, or to prevent tissue adhesion, or to prevent scarring.

15. A method according to claim 13
wherein the therapeutic agent includes stem cells, or antibodies, or antimicrobials, or collagen, or a gene, or DNA, or combinations thereof.

16. A device for implanting in an animal body comprising a device body and a coating on at least a portion of device body comprising a poly(anhydride ester) material or a derivative of a poly(anhydride ester) material.

17. A device according to claim 16, wherein the coating further includes a therapeutic agent comprising an anti-inflamatory drug; rapamycine and analogs, such as everolimus and biolimus; dexamethasone; M-prednisolone; interferon γ-1b; leflunomide; mycophenolic acid; mizoribine; cyclosporine; tranilast; biorest; tacrolimus; taxius; pacitaxel; or taxol; botox; lydicane; Retin A Compound; glucosamine; chondroitin sulfate; or Geldanamycin analogs 17-AAG or 17-DMAG.

18. A method of treating an animal comprising
providing a device as defined in claim 16, and
implanting the device in a tissue region of the animal.

19. A composition for application on to animal tissue comprising solution including a solvent and a poly(anhydride ester) material or a derivative of a poly(anhydride ester) material.

20. A composition according to claim 19, further including a therapeutic agent comprising an anti-inflamatory drug; rapamycine and analogs, such as everolimus and biolimus; dexamethasone; M-prednisolone; interferon γ-1b; leflunomide; mycophenolic acid; mizoribine; cyclosporine; tranilast; biorest; tacrolimus; taxius; pacitaxel; or taxol; botox; lydicane; Retin A Compound; glucosamine; chondroitin sulfate; or Geldanamycin analogs 17-AAG or 17-DMAG.

21. A method of treating an animal comprising
providing a composition as defined in claim 19, and
applying the composition to a tissue region of the animal.
